(19) United States
(12) Patent Application Publication
(10) Pub. No.: US 2022/0168367 A1
(43) Pub. Date: Jun. 2, 2022

(54) Destroying Cancer Cells While Maintaining Healthy Cell Integrity

(71) Applicant: Richard Postrel, Miami Beach, FL (US)

(72) Inventor: Richard Postrel, Miami Beach, FL (US)

(21) Appl. No.: 17/671,552

(22) Filed: Feb. 14, 2022

Related U.S. Application Data
(63) Continuation-in-part of application No. 15/954,573, filed on Apr. 16, 2018.
Application No. 15/954,573 is a continuation-in-part of application No. 15/808,563, filed on Nov. 9, 2017.
(60) Provisional application No. 62/595,043, filed on Dec. 5, 2017.

(51) Int. Cl. A61K 35/76; C12N 9/24; C12N 15/86

(52) U.S. Cl. CPC A61K 35/76; C12Y 302/01129; C12N 15/86; C12N 9/2402

(57) ABSTRACT

The present invention teaches a system and method for identifying, targeting and destroying cancer cells without harming healthy tissue. This invention uses a vector engineered virus to selectively seek out cells that are both hotter than normal with a pH factor that is lower than normal thereby identifying cells that are cancerous regardless of location. Cancer succeeds for two reasons: i) rapid growth and ii) failure of the body's immune system to recognize the aberrant cells. This novel approach of using a dual vectored virus creates a dominant preference for locating and attaching to cancer cells, eliminating the need for chemotherapy, radiation therapy, and most major surgeries.

The present invention teaches a system and method for identifying, targeting and destroying cancer cells without harming healthy tissue. This invention uses a vector engineered virus to selectively seek out cells that are both hotter than normal with a pH factor that is lower than normal thereby identifying cells that are cancerous regardless of location. Cancer succeeds for two reasons: i) rapid growth and ii) failure of the body's immune system to recognize the aberrant cells. This novel approach of using a dual vectored virus creates a dominant preference for locating and attaching to cancer cells, eliminating the need for chemotherapy, radiation therapy, and most major surgeries.

As cells progress from a normal state towards a cancerous state, the cancer's uncontrolled growth requires more energy (than normal cells), consuming all available oxygen, and resulting in compensatory metabolic pathways. The accelerated metabolic rates and compensating metabolic pathways produce a heat signature higher than those of neighboring cells which release excess amounts of H⁺ (resulting in a locally reduced pH) and a unique universal cancer signature.

Cancer's ability to evade the body's immune system is defeated by the present invention's power to “light up” or “highlight” cancer cells without marking uninvolved healthy cells thereby alerting the body's immune system to respond specifically to the targeted area of highlighted cancerous cells. The present invention directs the body's immune system towards cancer cells previously unnoticed by the immune system. Inserting foreign (e.g., viral) genetic material into the cancer cells highlights the cancer cells to alert the immune system to action in the virally infected zone. But one characteristic of many cancer cells is that as the cancer developed, the innate pathways in a cell that signal the systemic immune system to attack abnormal cells is inactive. With the present invention, cells on the periphery of a tumor, possibly not fully progressed to “cancer” are recognized as targets, possibly because their metabolisms have started to transform, but definitely because of the higher heat and decreased pH immediate to the tumor. Another ring of supportive cells immediately surrounding the cancer is also targeted because of the “cancer signature” local temperature increase and decreased pH. These cells immediately adjacent to the cancer will, when infected by the virus, be fully active in drawing a full immune response to the cancer site even when the cancer cells have mutated to evade recognition by the immune system. The viral particles themselves, concentrating in the area in and surrounding the cancer are recognized as alien (foreign to the organism) bio-material and will elicit another immune response separate from the infected cells' releases of immune chemokines. The activities of the virus and immune system responding to the viral infection/presence surround and encapsulate the tumor to attract and direct immune activity to the cancerous cells, precancerous cells, and a zone of healthy cells, e.g., about 1, 2, 3, 4, 5, or 6 cell diameters, adjacent to and surrounding the tumor to encapsulate the entire tumor in formation. Thus the anti-cancer treatment will result in an infection event. The targeted organism may, depending on the strength of infection, exhibit symptoms commensurate with infections caused by viral strains similar to the source strain used for engineering.

Cancer is not a single disease. Cancer arises in many different tissues, with a result that the plasma membranes of cancer cells do not express a cancer specific protein for universal recognition. One strategy has been to isolate an individual's cancer's cells in culture with the aim to form an antibody against that specific cancer to evoke an immune response targeting the cancer cells but sparing normal tissue. Numerous cultures producing antibodies that react with normal tissue must be discarded while cancer specific antibody cultures are grown and tested for reactivity against other healthy tissues. This is an arduous process. The present invention provides an alternative by engineering a virus that infects cells with two universal features of cancer cells.

If a cancer reoccurs, or the organism develops a subsequent cancer, the engineered virus useful in shrinking or eradicating the first cancer may be attacked by the memory components in the immune system and thus be unable to achieve full effect at the subsequent tumor site. Experience with the flu virus, e.g., a new flu vaccine used annually, offers the ability to have a second, third or fourth generation of anti-cancer engineered virion assemblies.

Cancer cells are faster growing than non-cancer cells. The faster growth requires an accelerated metabolism with a greater number of chemical reactions that produce heat. The accelerated metabolism also shifts metabolism to a rapid pathway that produces the cell's energy source, ATP, with a byproduct of lactic acid. Thus, cancer cells exist in an environment where temperature is elevated and acidity is increased.

Each cancerous cell thus presents with at least one early onset bio-nanomarker in the form of one or more metabolic differentials that have shifted to support the massively enlarged number of chemical reactions/interactions necessary to support the enhanced replication, or simply “hyperproliferation” that is characteristic of cells of the cancer group. Although different cancers may appear in disparate tissues and cancer cells may migrate from one tissue to another, at their root each cancer cell cohort involves a shift in normal metabolism from a lower to a higher metabolic rate, this shift being a universal characteristic of all hyperproliferating cancerous cells. As a cell transitions to become cancerous, it alters its metabolic pathways in various ways; down-regulating several, up-regulating others, possibly reinvigorating pathways used at an earlier time, for example during fetal development and turning off still others entirely. The present invention thus recognizes and can deal with cells that are not yet deemed cancerous but that have progressed substantially on a cancerous path.

As an example of a changed metabolic requirement, each time a cell divides it requires its own set of nucleic acids to construct a second complete genome. To accomplish this, the nucleic acid production pathway must be up-regulated. But the up-regulation of one pathway requires diverting nutrient availability within the cell to deprive other pathways of their normal resource pools favoring transformation towards a more opportunistic cancerous supportive metabolic function. Outcomes of these metabolic shifts include an increased release of H⁺ with a resultant drop in pH and increased release of small carbon containing molecules. Since all cancer cells are on their face abnormal, their activities, i.e., metabolism, will present diverse metabolic pictures, with the commonality of pathways supporting hyperproliferation. In view of these considerations, cancer can be thought of as a single disease—inappropriate hyperproliferation—but with several modes of expression that are supra-dependent on the initial metabolic status of the cell and stresses or pressures that make or cause the metabolic changes to occur that are necessary to support hyperproliferation. This invention teaches systems and methods for identifying, targeting and destroying cancer cells. As cells progress from a normal to a cancerous state their accelerated metabolic rates and adapted pathways generate amounts of H′ along with a higher heat signature that can serve as a targeting beacon for a specialized cell killing vectors such as a virus, preferably an engineered influenza virus. Influenza is a preferred virus at least because it frequently and continuously mutates to present variants no longer recognized by the previously infected immune system. These constantly appearing novel flu viruses require the updating and re-inoculation with flu vaccine which is done annually. When a flu virus is engineered to effect the present invention, a version that has not been recently active should be selected to serve as the strain to be engineered. When a virus other than a flu virus is engineered, the seed strain should be selected to avoid those that may have recently infected the target organism.

This production process is carried out in the cytoplasm and produces less ATP per glucose molecule, and also ends with lactate, a three carbon molecule, instead of the single carbon molecule, CO2. The metabolite, lactate, is a chemically energetic molecule whose energy is lost to the cell when the lactate is excreted using a slow but effective transport protein, monocarboxylate transporter protein (usually MCT4 or MCT 1). Lactate can be recycled by other organs in the body, e.g., the liver, to salvage the energy and carbon building capacities of the lactate molecule.

As mitochondrial ATP production is de-emphasized, cytoplasmic pathways using enzymes evolved for ATP production pathways become more active. Generally, in pathway activation, expression is accentuated for the newly needed enzymes and transport proteins. Activation often starts at the transcription level which progresses through messenger RNA to ribosomal synthesis of extra copies of the proteins necessary for the pathways.

Some proteins are up-regulated. Others are down-regulated. Many will feedback through the pathway or regulate activity of other functions or cell proteins. For example, pyruvate kinase M2 (PKM2) plays a part in the altered glucose metabolism characteristic of cancer. Inhibiting one or more such enzymes using a virus, a small molecule or biological inhibitor and/or ligand starvation or product feedback negative feedback may be used with the systems and methods of this invention.

When pyruvate kinase M2 (PKM2) interacts with phosphotyrosine-containing proteins, it inhibits their enzyme activities resulting in an increased availability of glycolytic metabolites the cell may then use to support and encourage cell proliferation. An alternate pyruvate kinase MI (PKMI), same gene but processed differently (alternative splicing) within the cell, does not share this outcome. It can therefore be said that favoring genetic processing conditions that increase PKM2 at the expense of PKMI is one factor supporting cancer development. While a mutation in the pyruvate kinase gene itself may affect splicing, a mutation in another gene or even an extracellular signal turning on or accentuating another path within the cell may be part of this cell's path to cancer.

As cells collect mutations, many will be culled by the organism's defenses which recognize damaged/unproductive cells and/or by the damaged cells activation of an internal self-sacrificing defense, such as apoptosis. But occasionally, a cell presenting a mutation leading towards a cancerous cell metabolism will evade these defenses and continue to reproduce. Several of the reproduced cells may be additionally mutated with each division. The same stress that may have encouraged the premiere mutation may encourage subsequent mutations and/or the premiere (or a subsequent) mutation may provide added stress encouraging still more mutations. Often cancer cells will carry a mutation that interferes with recognizing and repairing gene copying mismatches. Many of these mutations may still be removed by the organism's survival processes, but in rare, but significant to the organism, occasions multiple mutations can increase survivability of that cell line and continue to proliferate with continuously expanding mutations carried in the cell line's genome. At some point the collection of mutations and resultant metabolic responses will be sufficient to escape organismal control and will favor proliferation over the function the organism would like that cell type to perform.

### SUMMARY

Cancer cells present as a disease characterized by an undesired expression of numerous traits, particularly traits leading to a rapid cell division. A cell's life can be defined as the sum of all its chemical reactions. Since cancer cells differ from normal cells, their chemical reactions (aka metabolism) must, by definition, also differ.

Cancer cells arise from diverse tissues and from many, many differentiated cell types, but at the root of all cancers is that cell's increased rate of making new cells, that is: hyperproliferation. Every time a cell proliferates it splits to create two cells—each of which requiring its own membrane, cytoskeleton, nucleus, mitochondria and other organelles. This duplication requires the cell to accelerate synthetic pathways and several additional pathways that support accelerated synthesis. The resulting two cells will require a doubling of DNA for duplicated nuclei, additional membrane lipids and proteins to cover the increased surface/volume ratio, extra endoplasmic reticulum, golgi, mitochondria, lysosomes, etc. to be split between two cells during mitosis. Mitosis itself is a resource hungry process requiring a slew of catabolic and anabolic events. In essence, a metabolic push is necessary to provide an additional set of all cellular components and the temporary resources and energy necessary to divide the cell into two. This accentuated metabolism can be employed to guide intercourse between i) an involved party, e.g., an anti-cancer compound, probe, or other therapy, and ii) the cancerous, i.e., metabolically modulated cell(s).

Regardless of the cell type originating the cancer, all cancer cells will present an increased uptake of nutrient building blocks into the cell to support growth—and increased use of the nutrients (reactants) in various chemical reactions to make increased products. The products will include products useful for sustaining the cell and by-products such as waste chemicals and heat. While there are some common chemical waste products of metabolism, one ubiquitous product (since in general metabolism is exothermic) is an increased heat output.

Since cancer cells produce more heat than surrounding cells, increased temperature is a metabolism specific, local, let's say, “nanomarker”, that can be used to identify and target these cells for their destruction. While not an essential marker for all means of attacking cancer metabolism, heat can serve as a back-up confirmation or trigger signal for turning on natural innate and adaptive immunities and/or for making available one or more anti-cancer system(s) and method(s) in the identified cells.

The metabolic shift underlying increased metabolism de-emphasizes the production of ATP through the electron transport chain (ETC). Pyruvate is not fed into mitochondrial metabolism, but rater is converted to lactate and transported chiefly by monocarboxylate transporter 4 (MCT4) wherethrough H⁺ and lactate are delivered to the cell's exterior space. The H⁺ thus transported results in a decreased pH that coexists with the increased temperature.

Cancerous cells divide more frequently than normal cells and are produced and retained in an amount in excess of the optimal needs of the organism. Cancerous cells are not eliminated in the regular growth regulating processes in the body. Cells on the way to becoming cancerous, pre-cancerous or hyperproliferative cells, may form and grow at a pace in excess of the organism's needs, but exhibit some normal processes of cell maturation and death. Cells with the elevated growth rates will exhibit many metabolic characteristics similar to cancer cells, such as increased heat production and shedding acid, and so will be targeted by vectors, such as an engineered virus that focuses on cells in zones of elevated temperature and reduced pH.

A vector, for example a flu virus, is grown in culture conditions to favor binding and infective activity at elevated temperature. A vector, for example a flu virus, is grown in culture conditions to favor binding and infective activity at reduced pH. These engineering processes may be concurrent in the same culture or may be performed in parallel. When the vectors are engineered in parallel, the two parallel cultures are then combined in culture to favor both elevated temperature and reduced pH. The co-cultured viruses adapt to produce a culture that favors both low pH and elevated temperature. Engineering may include culturing the virus in a host environment that adapts the lipid envelope to increase melding with a targeted host at higher temperature or to decrease melding at non-elevated temperatures.

One recurrent theme in cancer therapies is a perceived benefit of early detection. However, because cancers arise from a variety of tissues and because cancers present as progressing diseases detection of specific cancers and selecting appropriate therapies is difficult. The universal recognition/attack on the cancerous cells is an important aspect in prompt response to a cancer upon detection.

The treatments of this invention can be used in conjunction with other advanced technologies. One such adjunct technology relates to detection of diseases, including cancers, through assaying volatile organic compounds (VOCs). Cancer cells, as a result of their altered metabolisms, produce patterns of VOCs characteristic to cancers in general. The specific cell type that is developing into, or has developed into, cancer will express, or emit characteristic, or signature patterns of VOCs. Use of such technology after a screen for VOCs from cancer's characteristic metabolic alterations can augment the universal treatment approach even applying universal therapy at a time very early in cancer's development. Treatment may then commence when an extremely small number of cells, not of sufficient quantity and influence on the organism to be noticed using screening methods currently practiced.

When additional VOC signature data is obtained, the cell type, and strength of the cancer(s) provide additional guidance for therapy. For example, in some circumstances, treatment may be ill-advised when the cancer is slowly developing and unlikely to seriously impact the individual's health or when treatment may be worse than the disease. But given the minimal impact of the present invention on healthy tissue, many cases where treatment is now ill-advised may rise up as stronger candidates for treatment.

Monitoring VOCs can serve as an adjunct, for example in the timing of treatments and in some cases for infecting with a differently engineered virus. Results from assaying VOCs may be used to indicate treatment efficacy. Or to help select the engineered virus. For example, VOCs may indicate degree of infection of the cancer cell tissues and/or may indicate that some cells are resistant to infection. An altered lipid content may sometimes be advantageous to better integrate into the cancer cells' plasma membranes. As a population of cancer cells is removed, the cells that remain, e.g., cells that evolved in a different p02 environment (perhaps shielded by outer cells in a tumor) may be more advantageously attacked with a better engineered virus, e.g., targeted at a less or more fluid membrane. Timing of repeated infections may be scheduled using VOC assay data.

### BRIEF DESCRIPTION

Cancer cells are differentiated by their altered and increased metabolisms. The altered metabolisms can serve as identifying markers, targeting markers and/or markers that signal or trigger a therapeutic intervention. The unbalanced metabolism can be used as an important marker identifying the altered cells.

The identification, targeting and triggering can include mechanics that are very high tech. For example, nanoparticles can be configured with nanosensor capabilities. These nanoparticles can be supplied in the vicinity of a tumor or may be applied more systemically, such as in blood or lymph vessels. One species of particle we can make has a form of nano-motor, or means of moving itself. These can be random or can be configured to be thermotaxic (move towards or away from a heat source) or chemotaxic (move along a chemical gradient, such as a pH gradient). Phototaxic (responsive to light—electromagnetic radiation, radio waves) sensors are another example, but these would be effective only close to the skin using ambient light or as secondary sensors responsive to a primary sensor that directs the secondary sensor to act at an identified location. Nanoparticles can also be configured as receivers of electromagnetic radiation. Nanoparticles compartmentalized for example by physical and/or chemical means can be queried to confirm location and if desired about the particle's surroundings. For example, the particle may report back an indication of temperature, pH, and/or other parameter programmed into the sensor. When the sensor is configured as an antenna, electromagnetic energy can be transmitted and converted to heat energy at the target location. Such particles may be used in conjunction with the viral-vector featured in the present invention.

Some organs present special difficulties to medical treatment. For example, the brain is sequestered away from normal delivery pathways behind an impenetrable layer of tightly packed cells known as the blood brain barrier (BBB). Although the BBB blocks harmful chemicals and bacteria from reaching the body's control center, in doing so the BBB blocks >90% of medicines delivered orally or intravenously into the circulation.

A natural or adapted virus, because of its infective structure can pass through the BBB and deliver encapsulated treatment throughout the brain. Because these infective agents are small and adept at entering cells, they are useful for delivering therapeutic agents, such as drugs, genetic therapies, etc., too hard to access tissues as well as those more freely accessible. Many viruses, including the SARS-CoV-2 virus incorporate proteins that facilitate crossing the blood-brain barrier. Human albumen proteins and fragments thereof are amongst a group of proteins the researchers have used to facilitate entry of other compounds, including viral particles into the brain. As a demonstration of the efficacy of viruses for therapy, researchers have injected a virus carrying genes encoding green fluorescent protein into the bloodstream of a mouse. In this case, the virus was carrying genes that encoded green fluorescent proteins. They observed that the virus infiltrated most brain cells and had the expected glowing effect.

While by no means all viruses inherently cross the BBB, viral engineering can make use of known methods such as used by the research referenced above to endow viruses with surface proteins adept at traversing this and other barriers.

Heat from Metabolism

Less technological applications of the invention are also available. Chemicals, especially lipid compositions, are heat responsive. Following the activation energy theories involved in completing a chemical reaction, including those facilitated by catalytic enzymes, chemical reactions are temperature dependent. Thermo-dependence is even more evident in enzymatic reactions where subtle temperature changes can induce profound changes in a protein's or RNA's folding and activity. According to these three-dimensional models, a complex molecule's binding site(s) require stability in the interactions of multiple hydrophobic and hydrophilic parts of a molecule. At a low energy state (lower temperatures), the molecule's kinetic energies will be insufficient to dislodge hydrophobic and e.g., hydrogen bonds that maintain a three-dimensional shape conducive to the catalyst presenting a ligand's reactive site(s) to another reactant. Increased temperature can increase random kinesthesia in the molecule and disrupt the appropriate three-dimensional configuration. In the membrane, interactions between lipids changes with temperature as the constituents in the bilayer present with a more solid or more melted form. The melted state of the membrane or a portion thereof (e.g., disordered or raft portions) can govern its ability to meld with other lipids or present integral membrane proteins.

Molecular biologists have several decades experience using temperature to change nucleic acid, folding, binding and activity and are adept at engineering sequences to fold or unfold at desired temperatures. Nucleic acids can be engineered to produce a protein of interest, including proteins whose range of temperatures where they are active is an engineering consideration, using available and improving software. Nucleic acids whose transcription, processing or translation is required to make the proteins can also be engineered for desired temperature dependence. Protein shape is determined by its primary sequence of amino acids. But this sequence folds and holds shape dependent on associative proteins, ligands in a binding or modifier site, temperature, hydrogen binding, salt, ionic strength, etc.

Experienced biologists, engineers, chemists, etc., have available technology including hardware, software, artificial intelligence, etc., that allows close approximation in silico of protein foldings, temperature, pH, lipid, osmotic, ionic pressure, ionic strength factors and how these affect relevant components, for example, a specifically designed or selected lipid mix may intercourse and blend into another, such as a virus and vesicle, virus and membrane, vesicle and protein, vesicle and membrane, etc.

Another important feature common to the metabolic shift of cancer cells is the decreased reliance on the ETC for making high energy phosphates, e.g., adenosine triphosphate (ATP). To make the ATP that is required in amplified amounts to support the increased metabolism that supports the hyperproliferation, cells switch metabolic paths to emphasize a glycosylation process that ends with lactate(−) and hydrogen ion (H⁺) as byproducts. The additional H⁺ ions depress the pH (a measurement indicative of H⁺ concentration). Another common byproduct is an increased abundance of various reactive oxygen species (ROS) such as H₂O₂ and .0₂-.

These chemical signatures can be used in addition to or as alternative to the heat signature given off by cancer cells for identification and targeting. The local pH can also be used as an activator or triggering mechanism extracellularly and/or intracellularly. ROS species are very reactive and therefore will have greater applicability as an intracellular activator, but in specific circumstances these can be used as an activator signal or as a switch signal to be amplified in an extracellular application.

Although not observed in every cancer cell type, the increased metabolism often results in a modified plasma membrane. Some modifications are for stability, such as slightly longer fat chains in the membrane to raise the lipid melting point to coordinate with the increased heat of metabolism. Most cells also have increased numbers of membrane transporters, e.g., to facilitate nutrient uptake and waste disposal; some cancer cells express binding or transport proteins not normally expressed in the neighboring more properly differentiated cells. In other instances, a transporter is found at elevated concentrations in the membrane to support the substantially increased needs to transport some raw nutrients, such as amino acids and/or glucose. While these may be available as secondary targeting or trigger mechanisms, the primary mechanism—increased need for certain chemical reactions within the hyperproliferating cell—is a fundamental mechanism underpinning the identifying, targeting mechanisms of this invention.

Any available targeting or delivery means known in the art can be used. For example, a virus, e.g., a DNA or RNA virus can be engineered to deliver a therapy to the target cell's interior. In the example of a reovirus, the activated ras oncogene renders the cell more prone to infection by a virus since the activated Ras system deactivates a cell's antiviral defenses. Such an engineered retrovirus or other vector know in the art is therefore a viable courier for a variety of therapeutic strategies to modulate intracellular metabolism. A phase 1/1 1 study of intravenous reovirus in patients with melanoma (MAYO-MC0672 (NCI trial)), which has been performed. In this study, patients received systemic administration of reovirus at a dose of TCID50 per day on Days 1-5 of each 28-day cycle, for up to 12 cycles of treatment.

Other cancers of interest for reoviral therapy include: pericutaneous tumors, prostate cancer, glioma, metastatic ovarian tumors, head and neck tumors, metastatic sarcomas, nonsmall-cell lung cancer, squamous cell carcinoma lung cancer, pancreatic cancer, fallopian tube cancer, metastatic melanoma, colorectal cancer, etc. These studies investigated reoviral advantageous infection of cells compromised by ras activation.

Recognizing that the plasma membrane is a lipid bilayer and has a mosaic of proteins, glycolipids, lipoproteins, sterols, glycoproteins, etc., the fluid mosaic membrane lipid bilayer model popularized in the 1970s has been updated to include a conceptual structure referred to as “lipid rafts”. Lipid rafts are believed to exist as constantly changing structural components floating in plasma membranes. Lipid rafts are believed to play an important role in many biological processes, especially signal transduction, apoptosis, cell adhesion and protein orientation and sorting. Membrane proteins and lipidated peptides, carbohydrates or proteins either reside in, form the boundary of or be excluded from such rafts, depending on the molecule's physical/chemical properties. Since membrane binding and transport of molecules and signals across the cell membrane is the means through which cells interact with their environment including neighboring cells, lipid rafts are understood to play critical roles in many biological processes including viral infections.

The plasma membranes of eukaryotic cells comprise literally hundreds of different lipid species. The bilayer has evolved the propensity to segregate constituents laterally. This segregation arises from dynamic liquid—liquid immiscibility and underlies the raft concept of membrane sub-compartmentalization. Eukaryotic membrane lipids are mostly glycerophospholipids, sphingolipids, and sterols. Mammalian cell membranes predominately comprise but one sterol, namely cholesterol, but the membrane comprises several hundred of different lipid species of glycerophospholipids and sphingolipids. In glycerophospholipids the head group of varies, also the bonds linking the hydrocarbon chains to glycerol, and the length and location and degree of saturation fatty acids provide distinguishing molecular features including how they sort amongst each other. Similarly, sphingolipids have the combinatorial propensity to create diversity by different ceramide backbones and, above all, at least 500 different carbohydrate structures at the head groups of the glycosphingolipids. Cholesterol interacts preferentially, although not exclusively, with sphingolipids due to their similar carbon chain structure and the saturation of the hydrocarbon chains. Although not all the phospholipids within the raft are fully saturated, the hydrophobic chains of the lipids contained in the rafts are more saturated and tightly packed than the surrounding bilayer. Cholesterol then partitions preferentially into the lipid rafts where acyl chains of the lipids tend to be more rigid and in a less fluid state. Cholesterol is the dynamic “glue” that holds the raft together.

Molecule for molecule, cholesterol is often close to half the cell membrane molecules. But, since it is smaller and weighs less than other molecules in the cell membrane, it makes up a lesser proportion of the cell membrane's mass, generally ^{˜}20%. Cholesterol is also found in membranes of cell organelles, where it usually makes up a smaller but still significant proportion of the membrane. For example, the endoplasmic reticulum, which is involved in making and modifying proteins, is but 6% cholesterol by mass and the mitochondria, comprise about 3% cholesterol by mass.

Below the melting temperature (Tm), the membrane is gel like. The presence of cholesterol prevents ordered packing of lipids, thus increasing their freedom of motion, or in other words increasing membrane fluidity. Above this Tm (dependent on lipid content, especially cholesterol), the membranes are in liquid disordered state, the rigidity of cholesterol ring reduces the freedom of motion of acyl chains (trans conformation tends to increase order and help define the rafts. The decreased fluidity and higher order allow for a stronger resistance to disrupting influences such as polar molecules and thus decreases permeabilities to especially foreign substances such as water and nitrogen and oxygen containing compounds.

Without cholesterol, cell membranes would be too fluid, not firm enough, and too permeable to many molecules. Because the fatty acids are longer and more saturated (straighter), they aggregate more, which cholesterol also helps. That ordered part of the membrane is also thicker, making it better suited for accommodating certain proteins. Since the fatty acids in lipid rafts are longer, raft phospholipids move in sync with the phospholipids on the opposite side of the membrane. In the disordered portions of the membrane, the phospholipids on one side of the membrane move independently of those on the other. By stabilizing certain proteins together in lipid rafts, cholesterol is important to helping these proteins maintain their function.

Lipids, e.g., glycolipids such as a glycerolipid that has one fully saturated chain and one partially unsaturated chain could function as a surface-active component, a hybrid lipid or a linactant. These linactants would lower the line tension between domains by occupying the interface, with the saturated anchor preferring the ordered raft and the unsaturated fatty acid interacting with the less ordered lipid environment. Small finite-sized assemblies of disordered and ordered lipid domains separated and stabilized by these hybrid lipids could be expected to form as equilibrium structures. In the viral envelope especially but even in the plasma membrane with its generally lower protein content proteins, especially with multiple transmembrane domains such proteins should also act as linactants. Several protein structures would be ideally suited for this purpose. For instance, proteins that have both a GPI anchor and a trans-membrane domain have been identified, in which the GPI anchor could be raft-associated with the trans-membrane domain facing the non-raft bilayer. Another such protein is the influenza virus M2 protein, which seems to occupy the perimeter of the raft domain that forms when the virus buds from the plasma membrane. N-Ras has also been proposed to act as a linactant in the cytosolic leaflet of a raft.

Many studies indicated that membrane rafts must play an important role in the process of virus infection cycle and virus-associated diseases. Many viral components or virus receptors are exclusive to or concentrated in the lipid raft ordered micro-domains.

Viruses have been divided into four main classes, non-enveloped RNA virus, enveloped RNA virus, non-enveloped DNA virus, and enveloped DNA virus. General virus infection cycle is also classified into two sections, the early stage (entry) and the late stage (assembly and budding of virion).

Several studies have demonstrated the localization of viral structural proteins in membrane rafts and the effects of raft-disrupting agents (mainly removing reagents and synthesis inhibitors of cholesterol) in the replication processes of several viruses, including retroviruses (Retroviridae), RNA viruses (classified into Picornaviridae, Caliciviridae, Astroviridae, Reoviridae, Flaviviridae, Togaviridae, Bunyaviridae, Coronaviridae, Rhabdoviridae, Arenaviridae, Filoviridae, Orthomyxoviridae, and Paramyxoviridae), and DNA viruses (classified into Parvoviridae, Papovaviridae, Adenoviridae, Herpesviridae, Hepadnaviridae, and Poxviridae).

Initial viral infection arises via endocytosis or by injection of viral proteins and genes directly into the cytoplasm, by fusion of the viral envelope or by destruction of the viral capsids. Transcription and replication of DNA viruses except poxviruses generally happens inside the nucleus, whereas those of RNA viruses occur in the cytoplasm. However, influenza viruses are exceptional as RNA viruses with at least a major genome duplication occurring after transport to the target host cell nucleus. Before, after and during the transport and duplication processes, the innate immunity of the cell can act on the viral proteins and vRNA.

Once the progeny viral components have been produced, fragments are transferred to some organelles or to the plasma membrane, where formation of the progeny virus is processed by assembly and/or budding. Based on the viral outer boundary structure, virus particles are classified into enveloped viruses (Herpesviridae, Hepadnaviridae, Poxviridae, Flaviviridae, Togaviridae, Retroviridae, Bunyaviridae, Coronaviridae, Rhabdoviridae, Arenaviridae, Filoviridae, Orthomyxoviridae, and Paramyxoviridae) and non-enveloped viruses (Parvoviridae, Papovaviridae, Adenoviridae, Picornaviridae, Caliciviridae, Astroviridae, and Reoviridae). Influenza viruses belong to the grouping of enveloped viruses.

The envelope of these virus particles is acquired from the plasma membrane of the cell surface, Golgi apparatus, and/or endoplasmic reticulum (ER) by budding off these membranes. Influenza viruses, which are highly transmittable pathogens of severe acute respiratory symptoms in various animals including human beings, internalize into host cells through multiple pathways including clathrin-independent and caveola-independent endocytosis after binding of the virus to a terminal sialic acid linked to glycoconjugates on the cell surface via viral surface glycoprotein, hemagglutinin. After transportation of the virus to late endosomes, low-pH-dependent conformation change of hemagglutinin induces membrane fusion of the viral envelope with the endosomal membrane. Then viral ribonucleoprotein complexes (RNP) including the viral genome are released to the cytoplasm of host cells by proton influx of viral ion channel M2 protein that requires binding with cholesterol. Influenza virus particles consist of the viral RNP with an envelope that includes two spike glycoproteins, hemagglutinin and neuraminidase (NA), and ion channel M2 protein on the outer surface and internal MI protein and nonstructural NS2 protein on the inner surface. Membrane rafts are associated with the transmembrane domains and cytoplasmic tails of hemagglutinin and NA, with the short transmembrane domains of M2, and with NP but not with MI. Domains of hemagglutinin and M2 contain palmitoylated cysteine residues that can associate with lipids and cholesterol in rafts. Although these domains of NA are essential for the association with rafts, there is no evidence that NA possesses palmitoylated residues.

During the budding of enveloped viruses from the plasma membrane, the lipids are not randomly incorporated into the envelope, but virions seem to have a lipid composition different from the bulk host membrane. The virion envelope appears to be determined both by the virion protein content helping to order and select to surrounding lipids AND the presence and availability of specific lipids from which to extract. Although the cell and the culture conditions in which the progeny viruses are produced is a significant factor in determining lipid content and, to an extent, its Tm, the proteins present or deleted from the virus and mutations on these proteins as well as cell membrane lipid content and cell membrane proteins assisting in ordering the raft portions of the membrane are important factors.

Since the virus must contact the target cell before infecting it, recognizable features are used by viruses to attach to and gain entry into their targeted cell. Any surface feature including, but not limited to: a membrane protein, a meldable lipid blend, a specialized raft, a glycoprotein, a glycoprotein, and/or any portion or fragment thereof, etc., might be recognized by a targeting virus. Viruses may be engineered using molecular biology and/or mutated or adapted using for example serial culture to obtain viruses that recognize one or more selective feature.

All animal viruses must traverse cells' plasma membranes for access to the infected cell's machinery to propagate the virus. Cell entry occurs by membrane fusion (in enveloped viruses such as the flu). Although a protein rich capsid represents the outermost structure of naked viruses, it is surrounded by a targeted host cell-derived membrane in the case of enveloped viruses. Virus replication is a multi-stage process inside the respective host cell before viruses release to the environment to infect additional cells. Accordingly, to act as infectious agents, viruses must cross the host cell boundary at least twice during a replication cycle, once when entering and once for exit and distribution.

In enveloped viruses, entry occurs by fusion of the incoming virus with, and cell lysis during budding of the nascent virus across a cellular membrane. Virus entry is specific for susceptible host cells and depends on the viral surface proteins and receptors exposed on the target host cell membrane. Most cellular receptors are surface proteins of various functions, but sugars (i.e., one of the sialic acids for influenza virus) and lipids can function as receptors. Virus entry is often enhanced by nonspecific binding, thus increasing viral residence times at the cell surface. This non-specific binding is often achieved by glycosaminoglycans (e.g., heparan sulfate), which promotes cell attachment of many different viruses by ionic or electric charge interactions.

Simple plasma membrane fusion between envelope and cell appears to occur rarely—if it ever happens—and even viruses that can possibly fuse at the plasma membrane appear to commonly take an endosomal route. It appears that viruses rely on lipid rafts for entry. For example, it has been shown that several non-enveloped viruses go through a raft-dependent entry pathway that requires cholesterol. Lipid rafts are also involved in enveloped virus entry as can be inferred from preferred binding to raft-associated viral receptors (e.g., GPI-anchored or raft-associated trans-membrane receptors). Enveloped viruses also present with a requirement for entry based on raft integrity and cholesterol.

The “endosomal sorting complex required for transport” (ESCRT) components found in cell and organelle membranes clearly play an important role in viral infection, especially in the release of many, but certainly not all enveloped viruses, important exceptions being the herpesvirus human cytomegalovirus, human influenza virus, and respiratory syncytial virus. These viruses recruit alternative cellular machinery or may employ viral proteins facilitating membrane scission. For example, the influenza virus M2 protein comprises an amphipathic helix that is both necessary and sufficient for vesiculation in vitro and generally for influenza virus budding in tissue culture.

Influenza M2 is a trans-membrane protein that self-associates to become a homotetramer providing proton-selective (W) ion channel activity through the virion membrane. M2 binds to low cholesterol (lipid disordered) membrane regions to induce a positive curvature. M2 preferentially sorts to the phase boundary of phase-separated vesicles causing extrusion of the lipid ordered (10) domain, dependent on the presence of the amphipathic helix. The M2 pore localizes to the neck of influenza virus buds in virus-producing cells. Mutation of its amphipathic helix causes late budding arrest similar to late domain mutations in other enveloped viruses. Apparently, M2 serves an analogous function as the ESCRT-III/Vps4 complex in other viruses. The influenza virus membrane is enriched in cholesterol and is more lo than the surrounding plasma membrane thereby creating line tension at the phase boundary demarcating the viral bud. M2 preferentially sorts at this phase boundary and apparently modulates line tension through lipid membrane interaction of its amphipathic helices.

As alluded to above, lipid rafts appear as subdomains of a cell's plasma membrane. The rafts comprise elevated concentrations of cholesterol and glycosphingolipids. They exist as distinct liquid-ordered regions of the membrane that are resistant to extraction with nonionic detergents. Lipid rafts generally contain 3 to 5-fold the amount of cholesterol found in the surrounding bilayer. The lipid rafts are enriched in sphingolipids such as sphingomyelin, which is typically elevated by 50% in comparison to the disordered plasma membrane regions. As a result, phosphatidylcholine levels are decreased leaving similar choline-containing lipid levels between the rafts and the surrounding plasma membrane.

Each individual raft is small in area, but the many rafts constitute a relatively large surface of each plasma membrane. To exist as a demarcated entity each raft must have a distinguishing protein and lipid composition different from the disordered lipid membrane through which it floats, but all rafts all rafts of a cell are not mandatorily identical in terms of either the proteins or the lipids that they contain.

pH balance is extremely important to the biochemical reactions that define the gamut of metabolic processes—and cell and organism physiology as a result. As a rule, blood will be in a slightly alkaline range of about 7.35 to 7.45. Management of the pH is so important that the body's primary regulatory systems (especially breath, circulation, and renal controls) closely regulate the overall acid-alkaline balance and will counteract, on a system wide or whole organism basis, pH aberrations caused by local metabolic anomalies. The result is that the gross pH is generally maintained within a “normal” range irrespective of local stresses.

Certain viruses (including the rhinoviruses and coronaviruses that are most often responsible for the common cold and influenza viruses that produce flu) infect host cells by fusion with cellular membranes preferably modified by increased temperature and at low pH for optimal action in this invention. These are referenced as “pH-dependent viruses.” Viruses can exhibit similar temperature and/or pH sensitive selectivity through modification of the viral recognition proteins to bind, for example, an MCT4 or similar protein expressed on the surface of the metabolism-altered heat producing cells.

Drugs that increase intracellular pH (alkalinity within the cell) have been shown to decrease infectivity of pH-dependent viruses. Since such drugs can provoke negative side effects, the obvious question is whether more natural techniques can produce the same or an opposite.

Influenza virus, a member of the family Orthomyxoviridae that is an enveloped virus containing a genome comprising eight segments of negative-sense single-stranded RNA (ssRNA) has strains that are especially sensitive to pH for their target cell binding and thus can be used to preferentially target cells in low pH environs produced by cancer cells that skew metabolism towards lactic acid as a metabolic product. Influenza is a lytic virus which rapidly kills the host cell when the offspring virus are released. Since flu is a lytic virus the host cell genome is immediately incapacitated so that the cell can no longer divide to form offspring cancer cells. This contrasts with retro viruses like herpes and HIV which follow a lysogenic cycle, inserting viral reverse transcribed DNA into the host genome while the host remains viable. When the host cell divides, the lysogenic phase retrovirus remains incorporated into both new cells' genomes. But eventually the viral DNA is activated to produce large quantities of new virus particles whereupon that host cell ruptures (is destroyed) as the new particles are released.

However, copies of the viral genome remain dormant in the multiple divided cells with “sleeper” retro DNA. For this consideration, retrovirus is a stronger candidate for genetic engineering of cells to correct genetic flaws, while non-lysogenic viral infection is better suited for targeting and destroying invading, diseased or otherwise unwanted cells. Influenza A can swap one or more of its 8 RNA strands (PB2, PB1, PA, HA, NP, NA, M, and NS) with co-infecting viral particles or may undergo a drift mutation, perhaps a single nucleotide base that changes a single amino acid or results in early truncation, with a robust change in transmissibility, infectivity, and/or mortality. Influenza is therefore easier to engineer than many other virus formats.

Hemagglutinin on the surface of the flu virus is instrumental for binding to and infecting target host cells. The 2009 swine flu is particularly illustrative of this phenomenon. Hemagglutinin mutated to become more acid stable as this HINI virus shifted from swine to humans. This lowered the pH at which the flu hemagglutinin was activated. The activation process triggers an irreversible change in the hemagglutinin's shape that then fuses the virus and target cell. The pH of activation is known to vary amongst various flu viruses. Avian and swine viruses are generally activated at about pH 5.5-6.0 compared to a two-fold higher [H⁺] or pH about 5.05.5 predominant for human flu viruses. In the context of the 2009 pandemic, HINI swine viruses which were previously activated at pH 5.5-6.0 mutated to become activated at pH 5.5 at the pandemic inception and as the pandemic progressed, the activation pH of the HINI pandemic virus declined to 5.2-5.4. This mutation process can occur naturally as pH of the target changes or for purposes of the present invention culturing susceptible cells at decreasing pH levels, where targets may be selectively cultured to decrease their pH ranges for survival and growth or by switching the target cell line if preferred. Lowering the activation pH of the hemagglutinin may be one means of selectively targeting cells that favor a more acidic metabolism.

The infection process starts when hemagglutinin binds to a monosaccharide sialic acid present on the surface of the target host cell. Influenza viruses deliver their genomes into the nucleus as multiple single-stranded RNAs. Newly synthesized viral RNA will then exit the nucleus for assembly into virus particles on and with the plasma membrane. The viral envelope can thus be engineered by choosing the host cell used to manufacture the virus particle.

In the low-pH environment of the endosome, the hemagglutinin is activated by a conformational change triggering its membrane fusion activity. The viral membrane fuses with the limiting membrane of the endosome to release the nucleocapsid into the cytosol. Flu virus delivery of genetic material is rapid. The total infection period—from docking onto the cell's surface to the RNA entering the cell nucleus—is two hours. Influenza A because of its ability to mutate by both antigenic drift and shift is a preferred type of influenza virus for engineering select mutations in furthering this invention. In a low pH environment, the pH stabilized viral particle may facilitate development or may take advantage of tunneling nanotubes to pass infectious RNA to neighbor cells without necessity for forming an envelope.

As an illustration, a class A influenza virus, e.g., H3N2, is cultured in a receptive host cell. (The H refers to the form of hemagglutinin; the N refers to the form of neuraminidase; human viruses have been HI, H2 and H3 and NI and N2; HINI and H3N2 are most common infectious forms in humans. About 20 hemagglutinins are known, while neuraminidases have been seen in over 100 varieties.) The pH is gradually decreased with subsequent passaging. Attenuation is monitored to assure the virus remains infectious to human cells other than the cultured cell strain. In a preferred embodiment attenuation is observed at normal pH, but infectivity remains at elevated [H⁺].

The temperature is also increased in culture to affect the content of the viral envelope to favor assimilation into membranes at increased temperatures. Alternatively, the low pH stable virus is allowed to mix with liposomes with higher melting temperature to transfer the liposomic constituents to the viral envelope lipid coating.

The resulting infectious virus is again screened or tested for selective infection at depressed pH and elevated temperature. Such virus may be delivered to a patient as a treatment for cancer, to target hyperproliferating cells and/or as a prophylactic event to seek out and eliminate cancerous cells that have not yet been outwardly observed, such as being palpated as a tumor mass. Some portions of tumors enter a quiescent state, for example when encapsulated by extremely active cells on the periphery which may prevent adequate nutrients, 02, etc., from reaching internal cells. These cells are subject to attack and removal by the immune system when the peripheral cells are infected. The infected peripheral cells provoke a general inflammation mediated through cells' intrinsic immunity activities and innate immunity processes associated with interferon mediated paths. The initial responses may promote cellsuicide limiting viral replication, but also releasing cytokines that draw inflammatory cells to the site. The inflammation inducing cells secrete antibiotic substances, especially active oxygen compounds including, but not limited to: superoxide, hypohalites, and hydrogen peroxide.

These toxins will impact local cells underneath the peripheral cells. Additionally, viral induced stresses will promote intercellular bridging in the form of tunneling nanotubes (TNTs) that will transmit anti-viral activities to the neighboring cells, up to about five or six cell diameters distant.

Although HI, H2 and H3, and NI and N2 are the common human infecting hemagglutinins and neuraminidases respectively, others may mutate to be compatible with human cells as hosts and able to cause human disease and death. For example, the recent outbreak of bird flu was H7N9 killing several dozens of humans, but apparently was not able to replicate in a form transmissible from human to human. In another example, such virus with lytic potential but lacking transmission between untreated humans in contact with the recipient is prepared as a pH and heat targeting lytic vector. Influenzas B and C may be cultured and applied in the invention for similar considerations. An influenza A H5N1 virus, another drifted avian virus though weakly transmissible to humans, apparently requiring thousands of copies to infect a human can be extremely pathogenic as it may occasionally drift.

Influenza A viruses are especially capable of inducing the expression of cytokine and pro-apoptotic genes in infected cells. Pathogenicity, cell lethality, replication efficiency, and transmissibility of influenza viruses depend on both viral genetic and host factors.

Hemagglutinin protein binds receptors and mediates viral-cellular membrane fusion during viral entry is the primary antigenic target during infection. Hemagglutinin protein is a trimeric class I membrane fusion protein that sports in its ectodomain a membrane-proximal, metastable stalk domain that is capped with a membrane-distal receptor-binding domain. Hemagglutinin protein is readied for membrane fusion by cleavage of the hemagglutinin precursor into a fusioncapable hemagglutinin1-hemagglutinin2 complex. Some H5 and H7 hemagglutinin proteins can be cleaved by intracellular furin-like proteases to elicit systemic virus spread with enhanced virulence of such highly pathogenic avian influenza (HPAI) viruses.

Infection by influenza virus' hemagglutinin surface glycoprotein binds sialic acid-containing receptors on the plasma membrane of a target host cell. In general, H5N1 influenza virus hemagglutinin proteins bind preferentially to a(2,3)-linked sialosides. Whereas human-adapted influenza viruses bind preferentially a(2,6)-linked sialosides. A switch from a(2,3) receptor binding specificity to a(2,6) receptor binding specificity may be preferred in adapting avian influenza viruses for mammalian hosts.

Hemagglutinin proteins from different strains and subtypes vary in activation pH values with a range from ^{˜}4.6 to ^{˜}6.0. Hemagglutinin proteins from HPAI viruses normally exhibit an activation pH value at the higher end of the range ^{˜}6.0, while human seasonal viruses have lower pH activation values, ^{˜}5.0 or less. H5N1 influenza virus isolates cluster in a range of ^{˜}5.3 to ^{˜}5.9. For individual viruses grown in sequential culture genetic drift is an effective tool for directed mutation towards a desired activation pH range to match that of a target host cell. For example, in HI, H3, and H7 influenza viruses, mutations that alter the hemagglutinin activation pH have been associated with changes in virulence in mice.

After receptor binding and internalization during influenza virus entry, the hemagglutinin protein is triggered by low pH to undergo irreversible conformational changes that mediate membrane fusion, and initiation of cell lethal infection either through apoptosis or other cell death or through lytic release of virus.

An initial stage of immunity occurs within the cell under attack by a foreign (pathogenic) genome. Pattern recognition receptors (PRRs) recognize pathogen-associated molecular patterns (PAMPs) on invaders to initiate both the near instantaneous intracellular innate and the delayed and lasting adaptive immune responses. Toll-like receptors (TLRs) comprise an important set of PRRs where TLR activation initiates induction of interferons (IFNs) and cytokines active in both innate and adaptive immunity. Humans have at least 10 TLRs (appropriately numbered TLRs 1-10). The various TLR proteins bind different type targets, for example, TLRs 1 and 2 are involved in bacterial infections through their recognition of lipopeptides (1) and lipopeptides, lipoproteins and glycolipids (2); TLR3 recognizes double stranded RNAs and thus is preferentially effective against viruses. TLRs 7, 8 and 10 are activated in the presence of ssRNAs, especially of the types found in influenzas. TLR7 and TLR8 especially recognize GU of AU rich sequences of ssRNA viruses such as the Orthomyxoviridae family that includes influenza virus.

Compared with seasonal influenza virus HINI, highly pathogenic avian influenza virus H5N1 is a more potent inducer of TLR 10 expression. Influenza virus infection increases associated TLRs expressions which contribute to innate immunity through their sensing the viral infection. This leads to cytokine induction, especially proinflammatory cytokines and interferons. Since TLR 10 induction is more pronounced following infection with highly pathogenic avian influenza H5N1 virus compared with a less pathogenic HINI virus H5 influenzas are a preferred initiator of cell death.

However, although HINI viruses may be effective for infecting human cells, previous exposures to similar H/N epitopes may compromise access to target cells. Accordingly, it is advised to be cognizant of recent flu outbreaks that may have produced antibodies and other humoral reservoirs that might neutralize specific cell lines.

But as humans and other organisms have adapted to minimize and therefore better survive viral invasion, viruses also adapt to continue viral propagation. Among the 11 proteins encoded by influenza virus, the NSI protein has been shown to block the production of IFN-ß in infected cells. Such adaptations of an influenza virus allow it to evade host cell innate immunity, but are easily avoided in an engineered virus.

For example, influenza viral protein NSI serves to bind viral RNA with its RNA binding domain to shield it from contacting ssRNA sensitive TLRs and retinoic acid inducible gene-I (RIG-I) a protein recognizing dsRNA including looped ssRNAs that complementarily bind. When stimulated by binding RNA, the TLRs, RIGI and the like induce type I interferon production. Some NSI proteins also bind the tripartite motif-containing protein 25 (TRIM25) that works though the activation of RIG-I. NS1s apparently also can complex with RNA-dependent protein kinase (PKR) and inhibit it. Otherwise, PKR is activated by binding double-stranded viral RNA and causes translation arrest in the cell nucleus including inhibition of viral protein synthesis. As another defense, the influenza virus M2 protein can inhibit P581PK to inhibit protein synthesis, and arrest host cell apoptosis.

Influenza PB1-F2 with a serine at position 66 is especially adept at inhibiting type I interferon production. This PB1-F2 binds to and inactivates mitochondrial antiviral signaling protein (MAVS). PB1-F2 protein is also associated with the induction of apoptosis and has a synergistic effect on the function of influenza virus polymerases PA and PB2. PB2 can also bind and inhibit the interferon promoter stimulator 1 (IPS-I) that normally promotes IFN-ß production.

The invention modifies a natural infection process in recognition of the characteristic that cancers exploit an imperfection in our immune systems. The cancer cells arise from the organism's gene allotment and thus are native to the organism. Often mutated cells will stimulate an organism's immune defenses and will be eliminated. Unhealthy cells generally signal their ill health to cells around them or through secretions into the circulation. Immune responses are called in play to recycle the abnormal or diseased cell's components. But when this system of signaling or of recognizing aberrant cells fails, aberrant cancer cells may survive and proliferate. The proliferation may manifest to other cells, for example, by increased growth and depletion of nutrients which may cause the affected cells to sicken and to call in an innate immune response. The immune response may involve a general inflammation that in favorable situations may also eliminate the proximal cause weakening these cells. However, cancers may survive and continue to proliferate. Since the cancer's cells are native to the organism the adaptive or specific immune systems, additional therapies may be necessary to label these cells for immune attack. One embodiment of the present invention features a virus, such as an influenza derived virus to recognize a cancer cell and infect it to initiate immune responses.

The invention recognizes that not every cancer cell will be successfully infected. However, universal infection is not a requirement for attracting immune cells and/or inducing caspace dependent programmed cell death e.g., through necroptosis, pyroptosis, apoptosis, or caspace independent programmed cell death, e.g., autophagy, paraptosis, mitotic catastrophe, etc. Intercellular communications through chemical messaging and/or direct cytoplasmic connections between cells may share small macromolecules such as nutrients, RNA, signal peptides, etc., and larger components such as organelles as large as mitochondria, but they also share organism protective cell elimination or killing factors, spreading cell death to neighboring cells perhaps comprising a spherical shape as large as five or six cell diameters in radius.

It has long been recognized that cells in direct contact with one another have abilities for transfers of small molecules across the two membranes. One such structure cells use is the gap junction. Gap junctions are pore structures that directly connect the cytoplasm of two cells. This allows passage of small molecules, ions and electrical impulses to directly pass from the cytoplasm of one to the other. As a rule, a gap junction is available for molecules up to about 485 Daltons. Some gap junctions may impart an electrical charge selectivity. The interconnecting membranes remain impervious to most proteins, nucleic acids and virtually all organelles.

Gap junctions occur throughout body tissues including especially the heart muscle where they are instrumental in coordinated conductivity of the action potential throughout the heart muscle. Gap junctions are also present in other less active tissues including for example, skin. Gap-junction transmission is limited by the pore sizes of the proteins of the junction with small second messengers, e.g., inositol triphosphate (IP3) and Ca⁺⁺ representative of their activities. The transmission through gap junctions avoids losses into interstitial space.

Cells also communicate with cells to which they are not directly connected. Local secretions for example of lactic acid reduce pH and signal other cells of the local metabolism. Hungry cells can deplete fuels such as glucose thus providing signals to cells more distant from the blood supply than the hungry cell to stimulate angiogenesis by releasing their own angiogenetic compounds (a form of chemically mediated, non-contact, intercellular communication).

Stress is a strong stimulant for tunneling nanotube (TNT) formation. For example, malignant cells under ischemic stress release exosomes that stimulate TNT formation. TNTs are a type of direct contact intercellular communication constructed of tunnels that can occur between cells of some distance from one another, in some instances connecting cells as far as six cell diameters distant from one another. Stressed cells can reach out to near neighbor cells with a pipe-like structure or cytoplasmic tunnel between the cells. These tubular tunnels cam extend several cell diameters and can be of a size sufficient for transporting not only ions and small molecules, but substances as large as organelles, including, but not limited to: mitochondria, lysosomes, endoplasmic reticulum, Golgi, endosome and intracellular as well as extracellular amyloid R, etc. As a rescue mechanism, many cells have capacity to act as first responders to construct TNTs, a transient structure of a size compatible with in vitro organelle transfer from cell to cell as a rescue facilitator.

These tunnels are also capable of transporting viruses or parts thereof to neighbor cells. Several viruses increase proliferation through such TNT tunnels with the receiving cell acting to proliferate the viral particles, but also to initiate an immune within the receiving cell and subsequent secretion of cytokines to attract immune cells. These tunnels are thus capable of spreading a therapeutic virus from the maximally attractant cell to other nearby cancerous cells whose metabolisms may be tuned down, e.g., because tumor growth has deprived the cells of nutrients to support the cancer cell's enhanced metabolism.

TNTs by providing continuity between multiple cytoplasms of even non-contiguous neighbor cells allow transfer of cytoplasmic inclusions including ions, proteins, vesicles, organelles, infectious agents, etc. across multiple cell diameter and sometimes even around or bypassing intervening cells. The TNTs are labile structures that can form within a few minutes for short length TNTs or may form over several hours when the TNT bridges up to 5 or 6 cell diameters. TNTs may maintain intercellular connections for a range of time, from a few minutes up to several hours and even days. They may form rather transient cell-to cell connections in response to a single demand event or may maintain intercellular transmission of particles for extended periods, sometimes involving a reversal of flow between cells. TNTs are also dynamic and repeatable capable of establishing a cellular and subcellular network amongst cells that may persist for days and may connect between multiple cells to opportunistically provide access to a network of cells.

But TNTs are also capable of being put to use by pathogens to aid pathogenic proliferation with resultant harm to the host organism. The same routes used to carry beneficial nutrients, organelles and helpful cell-to-cell signals can also be turned to spread pathogens and harmful to the host messages between cells. The present invention takes advantage of such pathogenic transmission to carry the engineered pathogen from its original site to neighbor cells that the infected cells reach out to.

Useful physiologic functions for TNTTNTs include but are not limited to: cell-to-cell transfer of membrane patches, large structures such as membrane vesicles, organelles, electrochemical gradient, ion fluxes like signal transduction molecules (e.g., Ca++). For example, myeloid-linage dendritic cells and monocytes transmit calcium signals within seconds through their networked cells connected by TNTs. Stressed cells may stress neighbor TNT networked cells by sharing stressor or stressed induced compounds, especially reactive oxygen compounds (ROCs) and immune system activating substances. ROCs themselves although transportable through the TNTs are also inducers of TNT connections, reactive oxygen stress being a general result of stresses and a membrane disruptor attracting TNT forming proteins and lipids. Pathogens, by their nature, often induce the attacked cell to respond by producing toxic ROS. Reactive oxygens react to oxidize molecules at non-specific sites often denaturing or disabling biocompounds. Thus, initial attraction and attack by the heat seeking, pH sensitive infectious particle can recruit many cells to call in an immune attack, that not only targets the initially infected cell to preserve the organism, but also neighbor cancer cells.

In addition to ROS and immune secretions as signals that may be used individually or in combination to expedite TNTs, cardiolipin; hemeoxygenase-1; sirtuins; heat shock factors; including active fragments—including, but not limited to: HSP27, HSP40, HSP60, HSP60-HSPIO, HSP70, HSP90, HSPIIO, etc.; heat shock factor 1, including polymers; phosphorylated eukaryotic translation initiation factor 2a; activating transcription factor 6; histone deacetylase, cytochrome c; formylated peptides; intact in interstitium, clumped, polymerized, coordinated with or bound to lipids, carbohydrates other proteins, complexes and fragments of these and similar and analogous chemical signals may also be used to expedite or excite and guide TNT initiation, growth and production.

Pharmaceutical interventions are available to facilitate TNT production or to suppress TNT activities. Cannabinoids, including, but not limited to: anandamide (AEA), 2-arachidonoylglycerol (2AG), palmitoylethanolamide, noladin ether, O-arachidonoyl ethanolamine, oleoylethanolamide, plant and/or synthetic cannabinoids, including, but not limited to: cannabigerolic acid, cannabidiolic acid, A⁹-tetrahydrocannabinolic acid, cannabichromenenic acid, cannabigerovarinic acid, cannabichromevarinic acid, tetrahydrocanabivarinic acid, cannabidivarinic acid, cannabigerol, A⁹-tetrahydrocannabinol, cannabidivarin, cannabichromevarin, cannabichromene, cannabigerivarin, tetrahydrocannabivarin, Nisobutylamides, ß-caryophyllene, pristimerin, euphol, N-acylethanolamines, A⁸-tetrahydrocannabinol, guineensine, capsaicin, resiniferatoxin, HI-J-210, HI-J-331, JWH015, SATIVEX™ or its generic, dronabinol, nabilone, ajulemic acid, CP 55 940, CANNABINOR™ or its generic, methanandamide, THC-II-oic acid, TARANABANT™ or its generic, etc. may act in conjunction with sirtuins; HSPs, e.g., HSP90 (a CB2 chaperone), or through TRPVI to upregulate HSP27, HSP70, HSP90, etc. Any of these compounds or fragments may be provided intact, as a compound cleavable from a carrier portion, or attached to a carrier portion. One or more methylene groups—Cl-b—may be incorporated internally within the carbon chains to alter effects including, but not limited to: selectivity ratio for alternate receptors, half-life of activity, rate of delivery, level of activity, fluidity, toxicity, cost of production, etc. Such interventions may be used in conjunction with or as an improvement upon the baseline invention.

In several instances, molecular interactions of cannabinoid compounds with effector pathways for potentiating TNT formation are established. In many cases however, the precise pathway remains to be characterized. The pathways and interactions suggested herein relate to current understanding whose knowledge is not essential for practicing the invention. The suggested interactions are incorporated herein for guided understanding and should not be understood as dictum or as stages required for practicing the invention. The skilled artisan will also understand that these interventions are dose dependent. At therapeutic dosages affecting compromised cells with a reduced threshold for eliciting a distress call, less compromised cells will not see their thresholds breached; the needy cells will entreat one or more enabled neighbor cells for rescue through TNTs and/or other means. However, at higher dosages, the intervention(s) prompting the distress signal(s) would be expected to goad comprised cells to undergo actions associated with a poorer outcome, for example signaling their initiation of apoptosis. Signals resulting from these excessive dosages would have no reason to cause a TNT response.

When to main desired function of the virus is to induce cell death in contrast to many previous uses of viral infection to deliver a gene for genetic therapy, defective viruses, i.e., viruses lacking a full component of genetic material and associated proteins to reproduce more virus particles, and/or notably non-virulent viruses, e.g., viruses easily attacked by the host cell innate immunity, can be considered as viable or even preferred embodiments for use in the present invention. For example, flu viruses with one, two, three, four, five, six, seven, or even all eight RNA strands absent or modified to be incapable of expression or to result in a strong antiviral response and/or to lack viral defense against host cell antimicrobial defenses, even if no new viruses are made can still target the hyperproliferating cancer cell and by initiating cell apoptosis or other cell death, even non-productive lysis, serve the appropriate functions envisioned in this invention.

In addition to targeting the heat signature and decreased pH inherent in the lactate shift, targeting the MTC4 directly may be used for additional specificity and efficacy. The MCT4 being a transport protein has both intracellular and extracellular regions on respective sides of the plasma membrane. When an antibody is raised against MCT4 embedded in a membrane, Extracellular region specific anti-MCT4 antibodies have been raised. By incorporating Fab or antigen binding regions of such peptides onto a delivery instrument such as a liposome or engineered viral particle, delivery can be enhanced. For example, such antigen targeting fragment may be chimerized with a protein fragment of choice for embedding into the membrane of the delivery device. Recognition can be further improved by selecting binding fragments more strongly active in higher H⁺ and/or higher temperature environments.

Immune technology to purify and manipulate specific immune cell types with a goal of treating disease including specific cancers has evolved following President Nixon's declaration of war on cancer. Replacing or in conjunction with surgery, chemotherapy and radiotherapy, immunotherapy. Genetically engineered immune cells have been investigated as treatment options for HIV and several cancers. Lymphokine-activated killer cells, cytokine-induced killer cells, and natural killer cells, can mediate cancer regression with nonMHC restriction. Phase 1 trials using vaccine-induced expansion of tumor-specific effector

T Cells have Shown Promise.

In the absence of access to antibodies specific to a tumor's receptor molecule(s) such cellbased therapies though considered safe show limited efficacy. The present invention in immunelike targeting of MCP4 in conjunction with engineered elevated activities in higher temperature and lower pH environments (as made by cancer cells (and some pre-cancer cells) in general) provides enhanced concentration and activity at the desired sites of action. T cell receptors (antigen-specific t cell proteins and tools) are available from, for example, Astarte, Miltenyl, and other biomedical supply houses shown on the web, and/or may be made and improved by one skilled in such art. The general approach avoiding the individualization of each recipient's targeted cancer protein allows more rapid and less expensive therapeutic intervention.

Fueling Cells

All cells are living entities and as living things they require raw materials to maintain function, to grow and to reproduce. Lone cells can obtain their nutrition from the immediate surroundings. But in complex organisms, where the cell may be distant from the outside environment a delivery service is necessary. In larger animals the circulatory system is responsible for delivering and clearing food and waste. A blood supply transgressing through a system of tubes (blood vessels) is used. As the organism grows each part must be supplied with appropriate blood vessels for support. The formation of blood vessels requires migration and proliferation of endothelial cells. These endothelial cells must be fueled in order to form and maintain the circulatory system.

The circulatory system is also an information system. Blood can carry chemical messages to and from the cells it services. The message does not need a locational address. Since cells are in contact with the environment (interstitial space) they are constantly removing chemicals from the space and depositing chemicals into it. The tools on the cell surface that help transport chemicals across the cytoplasmic membrane are exposed to the interstitial space. If a molecule has characteristic affinity for one of these “receptors” it will associate as a ligand with the receptor. A receptor may have one of many functional characteristics. It may serve to allow viral attachment to the cell membrane. It may act enzymatically to change the ligand in a manner including, but not limited to: isomerization, cleavage, covalent attachment, internalization (carry across the membrane), initiate encapsulation, present the ligand in receptive form to another ligand or receptor, etc. The receptor often will induce further changes inside the cell to manage (or metabolize) in some way the molecule being brought into the cell. While often signals are molecules manufactured by one cell and delivered to another to instruct that cell what it should do, simply classical food molecules can serve as signals to upregulate the pathways needed to metabolize that type of molecule.

Most cells ingest the chemical mass and energy they need to grow and proliferate in a form of carbon they find easy to use, e.g., amino acids (proteins) and sugars (carbohydrates).

However, when the cell is behaving in a specialized manner, the cell often must alter its pathways to support the specialized needs. Or in the chicken-egg question, when the cell has activated surprising metabolic pathways, then the cell will by necessity be doing something distinct from “normal” cells.

For example, a growth signaling receptor protein when activated will initiate a signal cascade through to the cell nucleus to build food receptors and carriers and to transport these receptors and carriers to the plasma membrane. A sugar or amino acid then contacts the receptor and is carried inside. The carrier/transporter will initiate or activate an appropriate pathway inside the cell to metabolize the cargo. Perhaps the cargo is aminated or otherwise modified to divert to a less common metabolic pathway or to serve as an intracellular signal.

Acetyl COA

One popular branching point, i.e., a molecule that might be directed through several pathways is acetyl Co-A. Often acetyl co-A is produced from the degradation of carbohydrates and/or proteins. But, especially in circumstances where nucleic acid synthesis is required (e.g., rapidly proliferating cells or cells expanding mitochondria) mass) fatty acids may become a favored source of carbon.

Acetyl-CoA is a lipogenic precursor for many lipid molecules including, but not limited to: isoprenoid, cholesterol and fatty acids.

Another common precursor, oxaloacetic acid, which may also be directly exported from the TCA cycle, supplies pools of non-essential amino acids.

Countering Cancer's Metabolic Changes

Cancer cells are distinguished from other cells usually based on their loss of controlled functions normally carried out by that organ or cell type and by their hyperproliferation. While the hyperproliferation can be understood from the viewpoint of the cell whose fittest life mission is to grow and continue its cell lineage, from the organism's point of view this group of rogue cells is not supportive of the survival life of the large organism: First, these cells are not performing activities for the good of the whole organism. Second, these cells are wasting nutrients. Third, the increased volume occupied by these cells interferes with communication and other functions of the non-cancer cells. Fourth, these cells are consuming (wasting) resources that could be more advantageously used. And fifth, these cells may be exporting toxic or problematic metabolites requiring surrounding tissues to expend resources and effort in clean-up operation.

Since the cells are performing different, i.e., abnormal, activities one would have to expect that reactions within cancer cells will be different from those within normal cells. To put it simply, different outputs and behaviors will require different activities to achieve them. The hyperproliferative action of the mutating or mutated cells will require an abundance of nutrients. The increased rate of reactions will produce excess metabolites, possibly abnormal metabolites, and will result in excess heat from the exothermic reactions which predominate in the general nature of reactions.

The cells will also differ in the way they utilize intracellular and extracellular nutrients. Addressing these differences provides strategies for impeding tumor growth and tumor cell proliferation. For example, as the cells hyperproliferate, pathways for manufacturing purines and pyrimidines for nucleic acids must be accelerated.

Enhanced glucose uptake is a hallmark of several cancers and has been exploited in the clinic as a diagnostic tool through PET imaging of the glucose analogue 18F-deoxyglucose (18FDGPET). Moreover, in contrast to most normal tissues where much of the glucose is oxidized through the TCA cycle, in mitochondria, cancer cells preferentially convert glucose to lactate a three carbon molecule that retains and eventually removes energy unavailable for ATP synthesis. The fate of glucose inside cells is influenced by the enzymatic properties of the specific glycolytic gene products expressed. Expression of the M2 isoform of pyruvate kinase (PKM2) can contribute to the characteristic glucose metabolism of tumors and replacement of PKM2 with its splice variant PKMI cannot efficiently support biosynthesis and tumor growth. Pyruvate kinase appears to be an important gateway in glucose metabolism that can be critical for controlling cell proliferation.

The aversion of cancer cells to the ETC and the conventional oxidative phosphorylation pathway should be considered a requirement, not an anomaly of cancer cells. Remember that these cells were once considered “normal” cells but in their progression to the hyperproliferative state have had to alter normal cell functions. The hyperproliferation would be expected to change many metabolic pathways to support the new activities. These abnormal pathways would be expected to require abnormal raw materials or amounts of raw materials in the nutrients consumed or in the metabolic intermediates necessary to sustain the new way of life for the cell. It is thus wise to think of the altered metabolism, not as a symptom of cancer, but as links in the causative chain.

Most cancers are believed to present with a genetic abnormality. Several genes have alleles that support or initiate development of cancer. For example, greater than 50 cancers are associated with genes that increase cancer risk in individuals inheriting one or more copies from a parent. BRCAI and BRCA2 (associated with breast cancer), TP53 (Li-Fraumeni syndrome), and PTEN (Cowden Syndrome) are some well-publicized cancer risk genes. Several viruses, e.g., human cytomegalovirus, Epstein-Barr virus, human papillomavirus, hepatitis B virus, and hepatitis C virus are also genetic factors that increase cancer risk, i.e., events contributing to supporting a cancer cell's metabolic transformations.

An external event switching a gene on or off may initiate or contribute to the cancer cascade. If the organism is inattentive to the changing cell, the cell may be allowed to continue development to a cancerous status. But to support the change the cell will have to adapt. We have evolved means to halt the requisite adaptations. For example, either inherited or somatic mutations of TP53, a tumor suppressor gene for p53 protein, removes a brake on growth of abnormal cells and allows the metabolic transformations necessary for cancer cell proliferation to proceed. Another gene with tumor suppressive activity and whose mutation removes restraints on uncontrolled growth is CHEK2.

Some adaptations will be built in, in accordance with feedback loops that evolution has given us; some may involve additional mutations in the nuclear or mitochondrial genomes; some may be more complex evolved responses, for example, an epigenetic modification like methylation.

At the base of cell growth is metabolism and nutrition supporting the metabolism. The variety of underlying causes and adaptations supporting the initial events may require a variety of routes to counter the metabolic signature of a cancer cell. But all routes will to some extent address the abnormal metabolisms.

One simple course of treatment will be to support “normal” metabolism. That is to provide raw material (nutrients) supporting normal metabolism, for example to favor ETC activity. In concert with this can be a restriction on types of raw materials supporting the diverted or cancer enhanced or enhancing metabolic pathways. A more aggressive strategy may include inhibitors of one or more of these side pathways. When these cells are deprived of the environment in which they mutated and may have in fact contributed to, selective pressure will tilt against these cells in favor of the “normal” cells.

Nutrition can also be altered with a goal of supporting apoptotic activity and inhibiting cells that counter apoptosis. Such healthy cell supports may be used in conjunction with virally enhanced immune strategies.

Pyruvate and Lactate

Metabolic support of the immune response to infection of cancer cells can include multiple or alternative strategies depending on factors including, but not limited to: the cancer, stage of cancer, health of the host individual, cultural preferences, and availability of resources. Strategies that include addressing metabolic pathways involved in cancer maturation can enhance the immune effects of the present invention.

Pyruvate kinase catalyzes the last step of glycolysis, transferring the phosphate from phosphoenolpyruvate (PEP) to adenosine diphosphate (ADP) to yield adenosine triphosphate (ATP) and pyruvate. In mammals, two genes encode a total of four pyruvate kinase isoforms. The Pkrl gene encodes the PKL and PKR isoforms, expressed in the liver and red blood cells, respectively. Either the PKMI or PKM2 isoform encoded by the Pkm gene is found in cells. PKMI is found in many normal differentiated tissues whereas the PKM2 is expressed in most proliferating cells including all cancer cell lines and tumors tested. PKMI and PKM2 are derived from alternative splicing of a Pkm gene transcript by mutual exclusion of a single conserved exon that encodes 56 amino acids. Despite the similar primary sequences, PKMI and PKM2 have different catalytic and regulatory properties. PKMI appears always active, exhibiting high constitutive enzymatic activity. In contrast, PKM2 is less active, but is allosterically activated by the upstream glycolytic metabolite, fructose-I,6-bisphosphate (FBP). Unlike other pyruvate kinase isoforms, PKM2 can interact with proteins harboring phosphorylated tyrosine residues thereby releasing FBP which, in a feedback mechanism, reduces the activity of the enzyme. Low PKM2 activity, in conjunction with increased glucose uptake, facilitates use of glucose carbons into anabolic pathways derived from glycolysis. Also, PKM2, but not PKMI, can be inhibited by direct oxidation of its cysteine 358 as an adaptive response to increased intracellular reactive oxygen species (ROS).

Additionally, PKM2 expression in cancer cells has been associated with enhanced phosphorylation of the Hil on phosphoglycerate mutase 1 (PGAMI) by PEP. This pathway is an alternative route for pyruvate production but bypasses the generation of ATP via the pyruvate kinase step. This supports high rates of glycolysis. Replacement of PKM2 with the constitutively active isoform PKMI results in reduced lactate production, enhanced oxygen consumption, and a decrease in PGAMI phosphorylation. There also appears to be selection for PKM2 expression for growth in vivo. Alternatively, PKM2 expression may evidence selection against high pyruvate kinase activity and therefore against expression of PKMI. This rationale suggests that activation of PKM2 may impede cancer cell proliferation by interfering with regulatory mechanisms critical for proliferative metabolism.

It is expected that PKM2 activators will mimic the regulatory properties of constitutively active PKMI, thereby promoting high PKM2 activity regardless of the known mechanisms that cells use to decrease pyruvate kinase activity. Similar to results observed when PKM2 is replaced with PKM18, under standard tissue culture conditions, PKM2 activators had no significant effects on cell proliferation when tested across several lines. In contrast, when proliferation is assessed under hypoxic conditions C 1% 02), PKM2 activator treatment results in decreased rate of cell proliferation in comparison to DMSO-treated cells. And expression of PKMI in the presence of endogenous PKM2 has no effect on cell proliferation in standard tissue culture conditions, but inhibits proliferation under hypoxia to a similar degree as treatment with PKM2 activators. Replacement of PKM2 with PKMI also impairs cell proliferation under hypoxic conditions.

Cancer cells harbor genetic changes that allow them to increase nutrient uptake and alter metabolism to support anabolic processes, and interfering with this metabolic program is a viable strategy for cancer therapy. Altered glucose metabolism in cancer cells is mediated in part by expression of PKM2, which has specialized regulatory properties. Unlike its splice variant PKMI, which is found in many normal tissues, PKM2 is allosterically activated by FBP and can interact with tyrosine-phosphorylated proteins to release FBP and decrease enzyme activity.

Thus, growth factor signaling promotes decreased PKM2 activity and availability of glycolytic metabolites for anabolic pathways that branch from glycolysis. This suggests that activation of PKM2 might oppose the effects of growth signaling and interfere with anabolic glucose metabolism.

In this situation where pyruvate kinase activation has occurred, high pyruvate kinase activity would suppress tumor growth. Inhibiting pyruvate kinase may therefore provide a hotter target for the virus.

Countering Anti-Apoptotic Activities

The invention may incorporate actions and/or compositions the impact transcription, translation, cytoskeleton control or other factors that modulate the propensity or ability of proteins which disfavor apoptotic events in the cell. These proteins include, but are not limited to: Bc12, BclXI, BclxES, and Nip3. The virus may be engineered to include RNAs that decrease expressions and/or activities of one or more of such proteins.

Encouraging Pro-Apoptotic Activities

The invention may incorporate actions and/or compositions the impact transcription, translation, cytoskeleton control or other factors that modulate the propensity or ability of proteins which favor apoptotic events in the cell. These proteins include, but are not limited to: Bax, Bak, Bad, Bid, Bim, NoxA, Puma, proline oxidase, p53, cytochrome C, Hsp10, SMAC/DIABLO, apoptosis inducing Factor (AIF), endonuclease G, IAP inhibitor: omi/high temperature requirement protein A2 (HtrA2), adenine nucleotide translocator (ANT), cyclophilin D, peripheral benzodiazepine receptor, and procaspases. Viral engineering may consider enhancing apoptotic events.

Additional Safety

The viruses engineered for use in the present invention must possess capacity to infect cells and provoke an immune response. These viruses will preferably be attenuated to provoke adequate but not overwhelming immune attack. However, virus particles are capable of spontaneous mutation. Viruses also are known to swap genes and thereby change target cell, pace of infection, number of particles produced per infected cell, etc. Accordingly, preferred embodiments engineer viruses to incorporate at least one, but potentially a plurality of recognition sites for antiviral compounds, either existing and repurposed for this function or selected or designed specific to the binding site associated with the engineered virus. These recognition sites may also provide a two-tiered approach wherein the viral coat incorporates an engineered component that appears on the cell's membrane following fusion. This component can serve as a recognition site for elimination of the attacked cell, in some circumstances when the viral infection itself has not induced the robust immunogenic response to kill the cell and/or neighbor cells.

All patents and patent applications referenced herein are hereby in their entireties incorporated by reference.

What is claimed is:

1. A method for eliminating hyperproliferative cells in the body of an organism, said method comprising:
a) engineering a flu virus in culture to favor binding a target cell in a zone of elevated body temperature;
b) engineering a flu virus in culture to favor binding a target cell in a zone of elevated [H⁺];
c) continue engineering said flu virus to produce in culture a virion that favors both elevated temperature and elevated [H⁺];
d) introducing said virion into said body; and
wherein said virion focuses in one or more zones of elevated [H⁺] and temperature to increase anti-viral immune response in said one or more zones.

2. The method of claim 1 wherein said increased immune response involves infecting at least one hyperproliferative cell.

3. The method of claim 1 wherein said increased immune response is targeted at a zone of increased temperature and decreased pH in a zone immediate to a zone of hyperproliferating cells.

4. The method of claim 1 wherein said increased immune response involves cells that have not mutated towards a hyperproliferative metabolism, said involved cells proximal to a zone of hyperproliferating cells.

5. The method of claim 1 wherein said increased immune response is directed at the concentration or viral particles in said one or more zones of elevated [H⁺] and temperature.

6. The method of claim 1 wherein said increased immune response is directed at the concentration or viral particles surrounding said one or more zones of elevated [H⁺] and temperature.

7. The method of claim 1 wherein engineering a flu virus in culture to favor binding a target cell in a zone of elevated [H⁺] comprises introducing into said culture a virus engineered in culture to favor binding a target cell in a zone of elevated body temperature whose recognition protein is replaced with a second recognition protein harvested from a virus engineered though serial culture to favor binding a target cell in a zone of elevated [H⁺].

8. The method of claim 1 wherein engineering a flu virus in culture to favor binding a target cell in a zone of elevated body temperature comprises introducing into said culture a virus engineered in culture to favor binding a target cell in a zone of elevated [H⁺] whose recognition protein is replaced with a second recognition protein harvested from a virus engineered though serial culture to favor binding a target cell in a zone of elevated body temperature.

9. The method of claim 1 further comprising genetically modifying said flu virus to comprise a recognition protein capable of binding host cell MCT4 protein.

10. The method of claim 9 wherein said recognition protein capable of binding MCT4 protein comprises an intact or fragment of an anti-MCT4 binding protein.

11. The method of claim 10 wherein said anti-MCT4 binding protein comprises a Tcell receptor protein.

12. The method of claim 10 wherein said fragment of an anti-MCT4 binding protein comprises an antibody fragment.

13. The vector of claim 10 wherein said antibody fragment comprises at least one antigen recognition site that recognizes MCT4.

14. The method of claim 13 wherein said antibody fragment comprises a fab fragment that recognizes MCT4.

15. The method of claim 13 wherein said antibody fragment comprises a fab′2 fragment that recognizes MCT4.

16. The method of claim 1 further comprising:
repeating a)-c);
wherein said flu virus in a) and said flu virus in b) selected for said repeating differ in immunogenicity from said flu virus in original engineering in said a) and b) to a degree that cross immunity is rare or absent;
and introducing said second virion d) into said body of an organism that has previously received d).

17. The method of claim 1 further comprising:
a second repeating a)-c);
wherein said flu virus in said second repeated a) and said flu virus in said second b) selected for said second repeating differ in immunogenicity from said flu virus in original engineering in said a) and b) and in said second a) and b) to a degree that cross immunity is rare or absent; and introducing said virion into said body of an organism that has previously received d) and said second virion d).

18. The method of claim 17 wherein said flu virus in said repeating comprises a hemagglutinin (HA) subtype that differs from an HA subtype of said flu virus in original engineering in said a) and b).

19. The method of claim 17 wherein said flu virus in said repeating comprises a neuramindase (NA) subtype that differs from an NA subtype of said flu virus in original engineering in said a) and b).
